(19) United States
(12) Patent Application Publication
(10) Pub. No.: US 2004/0138610 A1
(43) Pub. Date: Jul. 15, 2004

(54) Active agent delivery device having composite members

(76) Inventors: Michel Cormier, Mountain View, CA (US); James Matriano, Mountain View, CA (US); Juanita Johnson, Belmont, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/745,995

(22) Filed: Dec. 24, 2003

Related U.S. Application Data
(60) Provisional application No. 60/436,590, filed on Dec. 26, 2002.

(51) Int. Cl. A61B 17/20

(52) U.S. Cl. 604/46; 424/447

(57) ABSTRACT

A device and method are provided for percutaneous transdermal delivery of a biologically active agent by applying a microprojection array to the skin of a person or animal with a system that has a composite applicator tip and/or a composite microprojection array system.

This application claims the benefit of U.S. Provisional Application No. 60/436,590, filed Dec. 26, 2002.

### TECHNICAL FIELD

This invention relates to administering and enhancing transdermal delivery of a biologically active agent across the skin. More particularly, the invention relates to a percutaneous delivery system for administering a biologically active agent through the stratum corneum using an array of skin piercing microprojections that have a dry coating of the biologically active agent. Alternatively, the biologically active agent is contained in a reservoir or matrix affixed to either surface of the microprojection array. Transdermal delivery of the agent is facilitated when the application of microprojections to the skin of a patient is done in a manner that increases the number of microprojections piercing the skin and increases the consistency of the depth of penetration of the microprojections.

### BACKGROUND

Active agents or drugs are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent into the bloodstream, while it assures no modification of the agent during administration, is a procedure that is difficult, inconvenient, painful and uncomfortable and which sometimes results in poor patient compliance.

In principle, transdermal delivery provides for a method of administering active agents that would otherwise need to be delivered via hypodermic injection or intravenous infusion. Transdermal agent delivery offers improvements in both of these areas. Transdermal delivery, when compared to oral delivery, avoids the harsh environment of the digestive tract, bypasses gastrointestinal drug metabolism, reduces first-pass effects, and avoids the possible deactivation by digestive and liver enzymes. Transdermal delivery also avoids the adverse effects of some active agents, such as aspirin, on the digestive tract. When compared to injections, transdermal agent delivery eliminates the associated pain and reduces the possibility of infection. In many instances, however, the rate of delivery or flux of many agents via the passive transdermal route is too limited to be therapeutically effective.

As is well known in the art, the term “transdermal” is a generic term referring to the passage of an active agent across skin layers. The term “transdermal”, as used herein, thus refers to the delivery of an active agent (e.g., a therapeutic agent, such as a drug, or an immunologically active agent, such as a vaccine) through the skin to the local tissue or systemic circulatory system without substantial cutting or penetration of the skin, such as cutting with a surgical knife or piercing the skin with a hypodermic needle.

Transdermal agent delivery includes delivery via passive diffusion as well as delivery based upon external energy sources, including electricity (e.g., iontophoresis), ultrasound (e.g., phonophoresis) and heat. Many transdermal agent delivery systems generally rely on passive diffusion to administer the active agent. The noted passive transdermal transport (or delivery) systems generally include an agent reservoir containing a high concentration of an active agent. The reservoir is adapted to contact the skin, which enables the agent to diffuse through the skin and into the body tissues or bloodstream of a patient.

While active agents do diffuse passively across both the stratum corneum and the epidermis, the rate of diffusion through the stratum corneum is often the limiting step. Many compounds, in order to achieve a therapeutically effective dose, require higher delivery rates than can be achieved by simple passive transdermal diffusion. Thus, in such instances, one or more of the above referenced external energy sources or active transport systems are employed.

Theoretically, the transdermal route of administration could be advantageous for the delivery of many therapeutic proteins, since proteins are susceptible to gastrointestinal degradation and exhibit poor gastrointestinal uptake and transdermal devices are more acceptable to patients than injections. However, the transdermal flux of medically useful peptides, proteins, polysaccharides, and DNA is often insufficient to be therapeutically effective due to the relatively large size/molecular weight of these molecules. Often the delivery rate or flux is insufficient to produce the desired effect or the agent is degraded prior to reaching the target site, for example while in the patient's bloodstream.

As is well known in the art, the transdermal agent flux is dependent upon the condition of the skin, the size and physical/chemical properties of the agent molecule, and the concentration gradient across the skin. Because of the low permeability of the skin to many active agents, passive transdermal delivery has had limited applications. This low permeability is attributed primarily to the stratum corneum, the outermost skin layer that consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal peptide and protein flux has been limited.

As stated, active transport systems use an external energy source to enhance agent flux through the stratum corneum. One such enhancement for transdermal agent delivery is referred to as “electrotransport.” This mechanism uses an electrical potential, which results in the application of electric current to a body surface to enhance transport of the agent through the stratum corneum.

There also have been many attempts to mechanically penetrate or disrupt the outermost skin layers thereby creating pathways into the skin in order to enhance the amount of agent being transdermally delivered. Early vaccination devices, known as scarifiers, generally included a plurality of tines or needles that were applied to the skin to and scratch or make small cuts in the area of application. The vaccine was applied either topically on the skin, such as U.S. Pat. No. 5,487,726 issued to Rabenau or as a wetted liquid applied to the scarifier tines, such as U.S. Pat. No. 4,453,926 issued to Galy, or U.S. Pat. No. 4,109,655 issued to Chacornac, or U.S. Pat. No. 3,136,314 issued to Kravitz. Scarifiers have been suggested for use in the delivery of intradermal vaccine in part because only very small amounts of the vaccine need to be delivered into the skin to be effective in immunizing the patient.

However, a serious disadvantage in using a scarifier to deliver an active agent is the difficulty in designing a system capable of delivering an exact predetermined dose. Also, due to the elastic, deforming and resilient nature of skin to deflect and resist puncturing, the tiny piercing elements often do not uniformly penetrate the skin and/or are wiped free of a liquid coating of an agent upon skin penetration.

Other devices that use tiny skin piercing elements to enhance transdermal agent delivery are disclosed in European Patent No. EP 0 407063A1, U.S. Pat. No. 5,879,326 issued to Godshall, et al., U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety. The noted devices use piercing elements of various shapes and sizes to pierce the outermost layer (i.e., the stratum corneum) of the skin.

The piercing elements disclosed in the cited references generally extend perpendicularly from a thin, flat member, such as a pad or sheet. The piercing elements in some of the devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

Generally, these systems include a reservoir for holding the active agent and also a delivery system to transfer the agent from the reservoir through the stratum corneum, such as by hollow tines of the device itself. One example of such a device that includes a liquid agent reservoir is disclosed in PCT Pub. No. WO 93/17754. The reservoir must, however, be pressurized to force the liquid agent through the tiny tubular elements and into the skin. The disadvantages of such devices thus include the added complication and expense of adding a pressurizable liquid reservoir and complications due to the presence of a pressure-driven delivery system.

Instead of a physical reservoir, it is also possible to coat the microprojections with the agent to be delivered and have this coating served as the reservoir, as disclosed in U.S. application Ser. No. 10/045,842, which is fully incorporated herein by reference. This eliminates the necessity of a separate physical reservoir and developing an agent formulation or composition specifically for the reservoir.

Thus, there is a need to control and increase the percentage of microprojections in an array that penetrate the skin as well as provide a way to control the variation in the penetration depth of the microprojections when the array is applied.

A device that has these capabilities will provide a means to deliver a dosage of active agent with less variation. Such a system is safer for the patient because the actual variation in the delivered dose is much smaller. In addition, the system is less expensive to manufacture because agent utilization can be more precisely estimated and wastage reduced.

The device and method of the present invention overcomes these limitations by transdermally delivering a biologically active agent using a microprojection array that is applied to the skin with a mechanical impact applicator, wherein the microprojection array and/or the impact application are adapted to increase the number microprojections in the array that actually penetrate the skin when the microprojection array is applied. In addition, the uniformity in the depth of penetration of the microprojections is also increased.

An effective agent delivery design for a coated microprojection array requires that the number of microprojections that penetrate the skin and the depth of penetration be as controlled and uniform as possible in order to effectively predict agent delivery. Variability in the percentage of microprojection penetration and the depth of penetration can significantly alter the total amount of active agent coating that is introduced into the skin and therefore significantly alters the amount of biologically active agent that is delivered from the coating.

One method to assist in the even and reproducible penetration of the skin by the microprojection array is to use a mechanical impact applicator to apply the microprojection array to the skin or other body surface. Such a device can be designed to apply a consistent and reproducible force to the microprojection array. This reduces variability between applications by the same user as well as reducing variability between users. Such a device includes an applicator tip that has an external surface that is designed to strike a portion of the microprojection array system and drive it into the skin with a predetermined and reproducible force. Several variations of designs and methods for an impact applicator are described in several pending U.S. applications, including application Ser. Nos. 09/976,798 and 09/976,763, which are fully incorporated herein by reference.

The present invention accomplishes this increase in the percentage of penetration and the uniformity of penetration by utilizing one of several configurations of the microprojection array and the impact applicator tip.

The invention calls for the use of a composite microprojection array and/or a composite impact applicator tip. A composite microprojection array consists of a two component layer attached to the skin distal surface of the microprojection array. In a preferred embodiment, the two component layer includes an annular ring of a compressible material surrounding a circular disk of a hard matrix material that is approximately the same diameter as the microprojection array.

The composite impact applicator tip consists of an annular ring of compressible material placed in recessed ridge located around the periphery of the impact applicator tip. The dimensions of the recessed ridge and the thickness of the compressible annular ring are such that the exposed skin distal surface of the compressible ring and the center portion of the skin distal surface of impact applicator tip are essentially co-planar.

Utilizing a microprojection based drug delivery system which includes one or both of these composite elements results in an increase in the number of the microprojections that penetrate the skin and also results in an increase in the uniformity of the depth of microprojection penetration.

The coating thickness is preferably less than the thickness of the microprojections. More preferably, the thickness is less than 50 microns and, even more preferably, less than 25 microns. Generally, the coating thickness is an average thickness measured over the coated microprojection area.

The most preferred agents are selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM-CSF, G-CSF, IL-10, glucagon, growth hormone releasing factor (GRF) and analogs of these agents including pharmaceutically acceptable salts thereof. Preferred agents further include conventional vaccines, recombinant protein vaccines, DNA vaccines, therapeutic cancer vaccines and small molecular weight potent drugs such as fentanyl, sufentanil, remifentanil, other opioid analogues and nicotine.

The coating can be applied to the microprojections using known coating methods. For example, the microprojections can be immersed or partially immersed into an aqueous coating solution of the agent, as described in pending U.S. application Ser. No. 10/099,604.

Alternatively, the coating solution can be sprayed onto the microprojections. Preferably, the spray has a droplet size of about 10-200 picoliters. More preferably, the droplet size and placement is precisely controlled using printing techniques so that the coating solution is deposited directly onto the microprojections and not on other “non-piercing” portions of the member having the microprojections.

In another aspect of the invention, the stratum corneum-piercing microprojections are formed from a sheet, wherein the microprojections are formed by etching or punching the sheet and then the microprojections are folded or bent out of a plane of the sheet. While the biologically active agent coating can be applied to the sheet before formation of the microprojections, preferably the coating is applied after the microprojections are cut or etched out but prior to being folded out of the plane of the sheet. More preferably, the coating is applied after the microprojections have been folded or bent out from the plane of the sheet.

### BRIEF DESCRIPTION OF THE DRAWINGS

The invention will now be described in greater detail with reference to the preferred embodiments illustrated in the accompanying drawings and figures, wherein:

FIG. 1 is a perspective view of a portion of one example of a microprojection array;

FIG. 2 is a perspective view of the microprojection array of FIG. 1 with a coating deposited onto the microprojections;

FIGS. 3A, 3B and 3B are graphical representations of several variations of impact applicator tips and microprojection arrays of the prior art (FIG. 3A) and the present inventions (FIGS. 3B and 3C);

FIG. 4 is a graph showing the variation in the depth of penetration when a microprojection array is applied to the skin by the use of several embodiments of the present invention; and

FIG. 5 is a graph showing the variation in perceived sensation when the several variations of the present invention are tested.

### MODES FOR CARRYING OUT THE INVENTION

Unless stated otherwise, the following terms used herein have the following meanings.

The term “transdermal” means the delivery of an agent into and/or through the skin for local or systemic therapy.

The term “transdermal flux” means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more biologically active agents may be coated onto the microprojections resulting in co-delivery of the biologically active agents.

The term “biologically active agent”, as used herein, refers to a composition of matter or mixture containing a drug which is pharmacologically effective when administered in a therapeutically effective amount. Examples of such active agents include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10) and glucagon.

It is to be understood that more than one active agent can be incorporated into the agent formulation(s) of this invention, and that the use of the term “active agent” in no way excludes the use of two or more such agents or drugs.

The agents can be in various forms, such as free bases, acids, charged or uncharged molecules, components of molecular complexes or nonirritating, pharmacologically acceptable salts. Also, simple derivatives of the agents (such as ethers, esters, amides, etc), which are easily hydrolyzed at body pH, enzymes, etc., can be employed.

The term “biologically active agent”, as used herein, also refers to a composition of matter or mixture containing a vaccine or other immunologically active agent or an agent that is capable of triggering the production of an immunologically active agent and that is directly or indirectly immunologically effective when administered in a immunologically effective amount.

The term “biologically effective amount” or “biologically effective rate” shall be used when the biologically active agent is a pharmaceutically active agent and refers to the amount or rate of the pharmacologically active agent needed to affect the desired therapeutic, often beneficial, result. The amount of agent employed in the coatings will be that amount necessary to deliver a therapeutically effective amount of the agent to achieve the desired therapeutic result. In practice, this will vary widely depending upon the particular pharmacologically active agent being delivered, the site of delivery, the severity of the condition being treated, the desired therapeutic effect and the dissolution and release kinetics for delivery of the agent from the coating into skin tissues. It is thus not practical to define a precise range for the therapeutically effective amount of the pharmacologically active agent incorporated into the microprojections and delivered transdermally according to the methods described herein.

The term “biologically effective amount” or “biologically effective rate” will also be used when the biologically active agent is an immunologically active agent and refers to the amount or rate of the immunologically active agent needed to stimulate or initiate the desired immunologic, often beneficial result. The amount of the immunologically active agent employed in the coatings will be that amount necessary to deliver an amount of the agent needed to achieve the desired immunological result. In practice, this will vary widely depending upon the particular immunologically active agent being delivered, the site of delivery, and the dissolution and release kinetics for delivery of the agent from the coating into skin tissues.

The term “microprojections” refers to piercing elements that are adapted to pierce or cut through the stratum corneum into the underlying epidermis layer, or epidermis and dermis layers, of the skin of a living animal, particularly a mammal and more particularly a human. Typically, the piercing elements have a projection length of less than 500 microns, more preferably, less than 250 microns. The microprojections typically have a width and thickness of about 5 to 50 microns. The microprojections can be formed in different shapes, such as needles, hollow needles, blades, pins, punches, other skin penetrating or piercing configurations and combinations thereof.

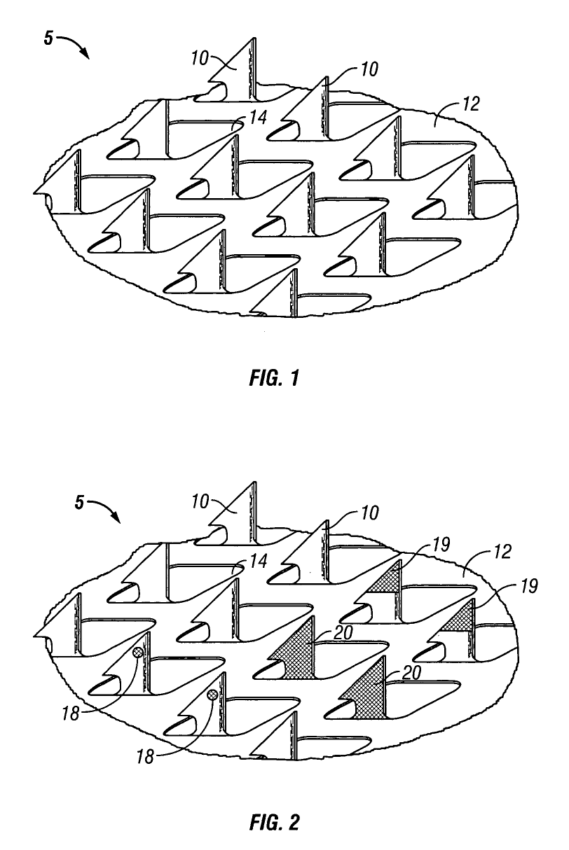

The term “microprojection array” or “microprojection member”, as used herein, refers to a plurality of microprojections arranged in an array for piercing the stratum corneum. The microprojection array may be formed by etching or punching a plurality of microprojections from a thin sheet and folding or bending the microprojections out of the plane of the sheet to form a configuration, such as that shown in FIG. 1. The sheet is typically circular in shape, but sheets having other shapes may be utilized. The microprojection array may also be formed in other known manners, such as by forming one or more strips having microprojections along an edge of each of the strip(s) as disclosed in Zuck, U.S. Pat. No. 6,050,988. The microprojection array can include hollow needles, which hold a dry pharmacologically active agent.

References to the area of the sheet or member and reference to some property per area of the sheet or member, refer to the area bounded by the outer circumference or border of the sheet.

The term “solution” shall include, not only compositions of fully dissolved components, but also suspensions of components including, but not limited to, protein virus particles, inactive viruses, and split-virions.

The term “pattern coating”, as used herein, refers to coating an agent onto selected areas of the microprojections. More than one agent can be pattern coated onto a single microprojection array. Pattern coatings can be applied to the microprojections using known micro-fluid dispensing techniques such as micropipetting and ink jet coating.

The term “microprojection array system”, as used herein, refers to at least the combination of the microprojection array, a backing membrane, various adhesive layers. If the system includes a ring of compressible material and a hard matrix disc, then the system is referred as a “composite microprojection array system”. If the system does not include a compressible ring and a hard matrix disc, it is referred to as a “standard microprojection array system”.

The term “composite applicator tip”, as used herein, refers to the tip of an impact applicator having a ring of compressible material peripherally attached to the skin proximal end of the applicator tip. If the applicator tip does not include a compressible ring, then it is referred to as having a “standard applicator tip”.

The compressible material preferably comprises a compressible foam having a compressibility, in a direction normal to the body surface being pieced, of more than about 50 μm. The compressible foam preferably comprises a closed or an open-cell foam.

The foam preferably comprises, without limitation, polyethylene, polyurethane, neoprene, natural Rubber, SBR, butyl, butadiene, nitrile, EPDM, ECH, polystyrene, polyester, polyether, polypropylene, EVA, EMA, metallocene resin, PVC, and blends thereof.

The term “microprojection based drug delivery system”, as used herein, refers to a combination of an impact applicator and a microprojection array system. The microprojection based drug delivery systems of the present invention include (i) a composite microprojection array system or (ii) an applicator having a composite applicator tip or (iii) a combination composite microprojection array system and an applicator having a composite applicator tip.

### DETAILED DESCRIPTION

The present invention provides a device for transdermally delivering a biologically active agent to a patient by the use of a microprojection based agent delivery system. The device includes a plurality of stratum corneum-piercing microprojections extending therefrom. The microprojections are adapted to pierce through the stratum corneum into the underlying epidermis layer, or epidermis and dermis layers.

The microprojections have a dry coating thereon that contains at least one biologically active agent. Upon piercing the stratum corneum layer of the skin, the agent-containing coating is dissolved by body fluid (intracellular fluids and extracellular fluids such as interstitial fluid) and released into the skin for local or systemic therapy.

FIG. 1 illustrates one embodiment of a stratum corneum-piercing microprojection member 5 for use with the present invention. FIG. 1 shows a portion of member 5 having a plurality of microprojections 10. The microprojections 10 extend at substantially a 90° angle from sheet 12 having openings 14. Sheet 12 may be incorporated into a delivery patch having a backing for sheet 12 and may additionally include an adhesive for adhering the patch to the skin. In this embodiment, the microprojections are formed by etching or punching a plurality of microprojections 10 from a thin metal Sheet 12 and bending microprojections 10 out of the plane of the sheet. Metals such as stainless steel and titanium are preferred. Metal microprojection members are disclosed in Trautman, et al., U.S. Pat. No. 6,083,196; Zuck, U.S. Pat. No. 6,050,988; and Daddona, et al., U.S. Pat. No. 6,091,975; the disclosures of which are incorporated herein by reference.

Other microprojection members that can be used with the present invention are formed by etching silicon using silicon chip etching techniques or by molding plastic using etched micro-molds. Silicon and plastic microprojection members are disclosed in Godshall, et al., U.S. Pat. No. 5,879,326, the disclosures of which are incorporated herein by reference.

FIG. 2 illustrates a portion of microprojection member 5 having a plurality of microprojections 10, some of which have a biologically active agent-containing coating 18, 19 or 20. According to the invention, these coatings may partially (coating 19) or completely (coating 20) cover the microprojection 10. The coatings are typically applied after the microprojections are formed.

The coating on the microprojections can be formed by a variety of known methods. One such method is dip-coating. Dip-coating can be described as a means to coat the microprojections by partially or totally immersing the microprojections into the coating solution. Alternatively, the entire device can be immersed into the coating solution. Coating only those portions the microprojection member(s) that pierce the skin is preferred. It is more preferable to coat only those portions of the microprojection member that come in contact with interstitial fluid.

By use of the partial immersion technique described above, it is possible to limit the coating to only the tips of the microprojections. There is also a roller coating mechanism that limits the coating to the tips of the microprojection. This technique is described in U.S. application Ser. No. 10/099,604, which is fully incorporated herein by reference.

Other coating methods include spraying the coating solution onto the microprojections. Spraying can encompass formation of an aerosol suspension of the coating composition. In a preferred embodiment, an aerosol-suspension having a droplet size of about 10 to 200 picoliters is sprayed onto the microprojections and then dried.

In another embodiment, a very small quantity of the coating solution can be deposited onto the microprojections 10, as shown in FIG. 2, as pattern coating 18. The pattern coating 18 can be applied using a dispensing system for positioning the deposited liquid onto the microprojection surface. The quantity of the deposited liquid is preferably in the range of 0.5 to 20 nanoliters/microprojection. Examples of suitable precision metered liquid dispensers are disclosed in U.S. Pat. Nos. 5,916,524; 5,743,960; 5,741,554; and 5,738,728; the disclosures of which are fully incorporated herein by reference.

Microprojection coating solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

The desired coating thickness is dependent upon the density of the microprojections per unit area of the sheet and the viscosity and concentration of the coating composition as well as the coating method chosen. In general, coating thickness should be less than 50 microns, since thicker coatings have a tendency to slough off the microprojections upon stratum corneum piercing. A preferred coating thickness is less than 10 microns as measured from the microprojection surface. Generally coating thickness is referred to as an average coating thickness measured over the coated microprojection. A more preferred coating thickness is about 1 to 10 microns.

The agent used in the present invention requires that the total amount of agent coated on all of the microprojections of a microprojection array be in the range of 1 microgram to 1 milligram. Amounts within this range can be coated onto a microprojection array of the type shown in FIG. 1 with sheet 12 having an area of up to 10 cm² and a microprojection density of up to 1000 microprojections per cm².

Preferred pharmacologically active agents having the properties described above include, without limitation, desmopressin, luteinizing hormone releasing hormone (LHRH) and LHRH analogs (e.g., goserelin, leuprolide, buserelin, triptorelin), PTH, calcitonin, vasopressin, deamino [Val4, D-Arg8] arginine vasopressin, interferon alpha, interferon beta, interferon gamma, menotropins (urofollotropin (FSH) and leutinizing hormone (LH), erythropoietin (EPO), GM-CSF, G-CSF, IL-10, GRF, glucagon, conventional vaccines and DNA vaccines.

In all cases, after a coating has been applied, the coating solution is dried onto the microprojections by various means. In a preferred embodiment, the coated device is dried in ambient room conditions. However, various temperatures and humidity levels can be used to dry the coating solution onto the microprojections. Additionally, the devices can be heated, lyophilized, freeze dried or similar techniques used to remove the water from the coating.

Other known formulation adjuvants can be added to the coating solution as long as they do not adversely affect the necessary solubility and viscosity characteristics of the coating solution and the physical integrity of the dried coating.

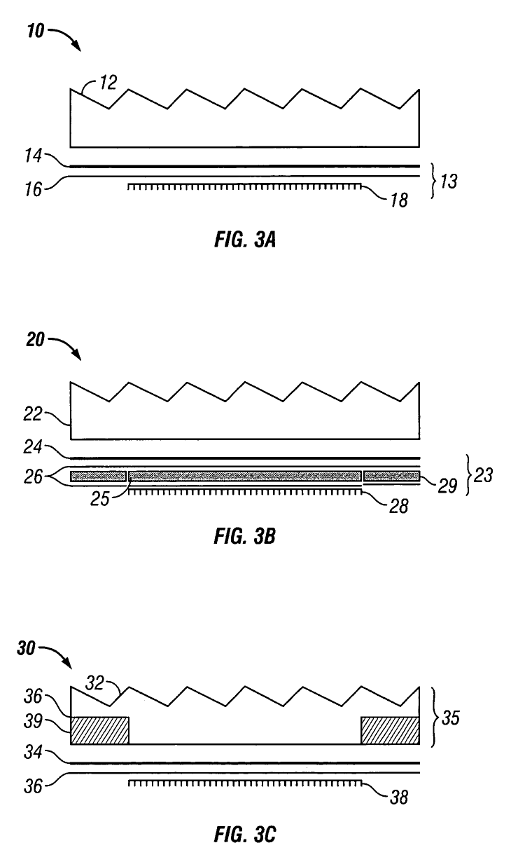

As indicated above, the present invention calls for the use of a composite microprojection array and/or an impact applicator having a composite applicator tip. Referring now to FIG. 3A there is shown a standard microprojection based drug delivery system 10, consisting of a standard impact applicator tip 12, which when utilized will strike the distal surface of the microprojection array system 13. Backing membrane 14 is attached via adhesive layer 16 to the microprojection array 18.

FIG. 3B shows microprojection based drug delivery system 20, a first variation of the present invention in which the impact applicator tip 22 is identical that shown in FIG. 3A. The composite microprojection array system 23 is composed of the backing membrane 24, which is attached to compressible foam 29 and hard matrix 25. Compressible foam 29 comprises an annular ring and encircles hard matrix 25 forming an essentially planar disk. Microprojection array 28 is attached to the hard matrix 25. Backing membrane 24 and microprojection array 28 are attached on opposite faces of the compressible foam 29 and hard matrix 25 by adhesive layers 26.

FIG. 3C shows microprojection based drug delivery system 30, a second variation of the present invention, in which the microprojection array system 33 is identical to microprojection system 13 shown in FIG. 3A. However, this variation includes a composite impact applicator tip 35. The composite tip 35 includes compressible foam 39 that is shaped as an annular ring formed around the periphery of the impact applicator tip 32 and disposed in a circular rabbit 36 formed in the edge of the tip 32. The depth of the rabbit 36 and the thickness of compressible foam 39 are essentially the same. Thus, the skin proximal surface of compressible foam 39 and tip 32 are essentially planar.

Though not shown, another embodiment of the present invention is a combination of the composite microprojection array system as shown in FIG. 3B used in conjunction with the composite impact applicator tip, as shown in FIG. 3C.

### EXAMPLES

The following examples are given to enable those skilled in the art to more clearly understand and practice the present invention. They should not be considered as limiting the scope of the invention but merely as being illustrated as representative thereof.

Testing was performed using one of three application systems. Referring to Table I, the first was a standard application system (element 10, FIG. 3A), which comprised a hard applicator tip and a standard microprojection array. The second application system consisted of a standard applicator tip and a composite microprojection array (element 20, FIG. 3B). The third application system consisted of a composite applicator tip and a standard microprojection array (element 30, FIG. 3C).

TABLE I

| FIG. No. | Applicator Tip | Microprojection Array |
|---|---|---|
| | standard | standard |
| | standard | composite |
| | composite | standard |

Each type of system was tested in both hairless guinea pigs (HGP) and human volunteers.

### Penetration and Homogeneity Test

In order to test for variations in the extent of puncturing that each of the above application systems produced, the three system configurations were tested on HGP's. Each type of system was applied to three HGP's, one system per animal. This resulted in the testing of nine animals in three groups with each group consisting of three replicates.

The actual systems tested consisted of a microprojection array having a microprojection length of 214 microns, having a diameter of 1.6 cm, an area of 2 cm², and having 585 microprojections per 2 cm². The systems were applied using an impact applicator which applied a force of 0.42 Joules in less than 10 milliseconds.

The systems were applied on the flank of the animal. The sites were manually stretch bilaterally just prior to application of the system. The stretching consisted of the application of two pairs of opposing forces with the pairs oriented at 90 degrees to each other. The systems were allowed to remain in place on the animals for 5 seconds and then removed. The sites were immediately stained with a 1% aqueous solution of methylene blue. Excess dye was washed away and pictures were taken of the sites. Each site was evaluated by judging the extent of staining based upon an evaluation of the photographs.

Since only those portions of the skin which are actually punctured are stained by the methylene blue, all staining evaluations are based on an evaluation of the color intensity at each microprojection puncture site. The deeper the microprojection penetrates the skin, the bigger the width of the puncture slit formed and the more intensely will the microprojection puncture site be stained.

The intensity of staining for each puncture site placed into one of four classes: no staining, slight staining, moderate staining, and intense staining. These classes were assigned numerical values of 0-3 respectively. For each of the above four classes, the percentage of the total application site which had staining which fell into each of the four classes was estimated. For example, if the staining of an application site were equally divided into each of the four intensity classifications, then that site would be given a ranking of 25% for intensity class 0, 25% for intensity class 1, 25% for intensity class 2 and 25% for intensity class 3. The resulting percentages for each intensity class as evaluated by each of the 3 judges were averaged together. The raw data is presented in Table II below:

TABLE II

| | Configuration | | | | | |
|---|---|---|---|---|---|---|
| | Applicator | Microprojection | Intensity | | | |
| System | Tip | System | Level 0 | Level 1 | Level 2 | Level 3 |
| A | Standard | Standard | 2.8 ± 2% | 13.3 ± 6.3% | 43.3 ± 8.8% | 40.6 ± 16% |
| B | Standard | Composite | 0 ± 0% | 2.8 ± 0.6% | 61.1 ± 6.8% | 36.1 ± 6.4% |
| C | Composite | Standard | 0 ± 0% | 3.9 ± 0.6% | 46.1 ± 2.4% | 50 ± 2.9% |

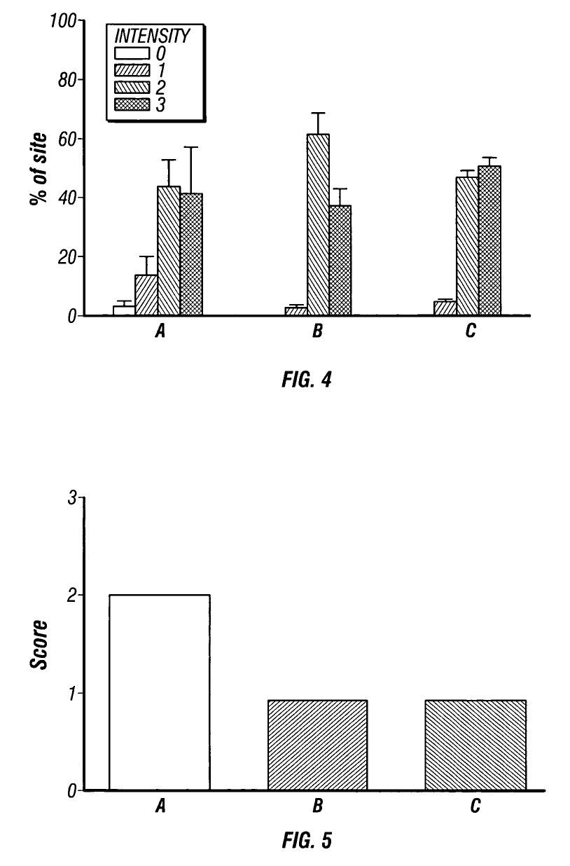

Each of the above percentages represents the average of three evaluations, each by a different person. The data is shown graphically in FIG. 4. The resulting averages for each system configuration are shown clustered together resulting in three clusters representing each of the three system configurations tested. Each cluster can contain up to four bars, each of the bars representing the percentage of the overall punctures sites that fell within one of the four classes.

A review of the data presented in FIG. 4 shows that cluster A had some regions of the puncture sites judged to be in intensity class 0. Cluster A also showed a relatively high percentage of puncture sites evaluated at intensity class 1, when compared to clusters B and C. Configuration A had a greater proportion of its puncture sites showing little or no staining when compared to clusters B and C.

Because neither cluster B, nor cluster C showed any puncture sites that were judge to be in intensity class 0, there are only three bars for these clusters. In addition, there was a shift away from intensity class 1 towards greater percentages in intensity classes 2 and 3. Cluster B shows the highest value for intensity class 3 at 61.1%. Cluster C showed no staining in intensity class 0, and a higher percentage in the intensity class 3, as compared to cluster B. These results demonstrate that system A produced a more heterogeneous puncturing of the skin than system B or System C.

### Reduction in Sensation

Additional studies were performed on human subjects in order to determine the effect of the various application system configurations on the perceived sensation of pain at the time of system application.

Each of the three configurations given in Table I above was tested on three human volunteers. Each volunteer had one each of the three systems applied. The systems were applied to different skin sites on the ventral forearm, alternating between one forearm and then the other. The systems were identical to those described above except the systems did not contain a microprojection array and the application sites were not stained.

Each volunteer was asked to rate their perception of the pain that they sensed when each of the three systems were applied. The ratings were assigned a value of 0 to 3 to represent the perceived pain as being no sensation, mild sensation, moderate sensation or severe pain.

The raw data from each of the three volunteers (V1, V2 and V3) for the sensation studies are given below in Table III.

TABLE III

| | | | Perceived | | |
|---|---|---|---|---|---|
| | Configuration | | Intensity | | |
| | Applicator | Microprojection | Score | | |
| System | Tip | System | V1 | V2 | V3 |
| | Standard | Standard | 2 | 2 | 2 |
| | Standard | Composite | 1 | 1 | 1 |
| | Composite | Standard | 1 | 1 | 1 |

The average for each system type was calculated and the results shown graphically in FIG. 5.

Please note, that because of the consistency of the data, the SEM for each average is zero and therefore no error bars are shown on the graph. The results indicate that a delivery system containing either a composite tip or a composite microprojection array system resulted in a lower level of sensation as perceived by the person on whom the system was applied.

Though the present invention has been illustrated with microprojection arrays having the biologically active agent coated there on, the principles of this invention can be equally applied to microprojection systems wherein the biologically active agent is contained in a reservoir or matrix affixed to either surface of the microprojection array. Illustrative are the reservoir and microprojection assemblies disclosed in U.S. Provisional Application Nos. 60/514,433 and 60/514,387, and PCT Pub. No. WO98/28037, which are incorporated by reference herein in their entirety.

While what are presently believed to be the preferred embodiments of the present invention have been disclosed, those skilled in the art will realize that changes and modifications may be made thereto without departing from the spirit of the invention, and it is intended to claim all such changes and modifications as fall within the true scope of the invention.

What is claimed is:

1. A composite microprojection system, comprising:
a microprojection member having a top surface and a skin distal surface, said microprojection member including a plurality of stratum corneum-piercing microprojections that project from said skin distal surface;
a substantially rigid matrix member disposed on said microprojection member top surface;
a compressible ring disposed on said microprojection member top surface and surrounding said rigid matrix member; and
a backing membrane disposed on said rigid matrix member and compressible ring.

2. The microprojection system of claim 1, wherein each of said plurality of stratum corneum-piercing microprojections has a length less than approximately 500 microns.

3. The microprojection system of claim 1, wherein each of said plurality of stratum corneum-piercing microprojections has a thickness in the range of approximately 5-50 microns.

4. The microprojection system of claim 1, wherein said rigid matrix and said compressible ring form a substantially planar disk.

5. The microprojection system of claim 1, wherein said compressible ring comprises a compressible foam.

6. The microprojection system of claim 5, wherein said compressible foam has a compressibility greater than 50 μm.

7. The microprojection system of claim 5, wherein said compressible foam comprises a substantially open-cell foam.

8. The microprojection system of claim 5, wherein said compressible foam comprises a substantially closed-cell foam.

9. The microprojection system of claim 5, wherein said foam comprises a material selected from the group consisting of polyethylene, polyurethane, neoprene, natural rubber, SPR, butyl, butadiene, nitrile, EPDM, ECH, polystyrene, polyester, polyether, polypropylene, EVE, EMA, metallocene resin, PVC, and blends thereof.

10. The microprojection system of claim 1, wherein said microprojection member is coated with a biocompatible coating, said biocompatible coating including at least one biologically active agent.

11. The microprojection system of claim 10, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts.

12. The microprojection system of claim 10, wherein said biologically active agent is selected from the group consisting of conventional vaccines, recombinant protein vaccines, DNA vaccines and therapeutic cancer vaccines.

13. The microprojection system of claim 10, wherein said biologically active agent is selected from the group consisting of fentanyl, sufentanil, remifentanil and nicotine.

14. The microprojection system of claim 10, wherein each of said plurality of stratum corneum-piercing microprojections includes in the range of 1 microgram to 1 milligram of said biologically active agent.

15. The microprojection system of claim 1, wherein said microprojection member includes a reservoir.

16. The microprojection member of claim 15, wherein said reservoir includes at least one biologically active agent.

17. The microprojection system of claim 16, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts.

18. The microprojection system of claim 16, wherein said biologically active agent is selected from the group consisting of conventional vaccines, recombinant protein vaccines, DNA vaccines and therapeutic cancer vaccines.

19. The microprojection system of claim 16, wherein said biologically active agent is selected from the group consisting of fentanyl, sufentanil, remifentanil and nicotine.

20. The microprojection system of claim 1, wherein said microprojection member includes an agent-containing matrix.

21. The microprojection system of claim 20, wherein said matrix is disposed proximate said top surface of said microprojection member.

22. The microprojection system of claim 20, wherein said matrix is disposed proximate said skin distal surface of said microprojection member.

23. The microprojection system of claim 20, wherein said matrix includes at least one biologically active agent.

24. The microprojection system of claim 23, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts.

25. The microprojection system of claim 23, wherein said biologically active agent is selected from the group consisting of conventional vaccines, recombinant protein vaccines, DNA vaccines and therapeutic cancer vaccines.

26. The microprojection system of claim 23, wherein said biologically active agent is selected from the group consisting of fentanyl, sufentanil, remifentanil and nicotine.

27. A composite microprojection system, comprising:
a microprojection member having a top surface and a skin distal surface, said microprojection member including a plurality of stratum corneum-piercing microprojections that project from said skin distal surface, said microprojection member being coated with a biocompatible coating, said biocompatible coating including at least one biologically active agent;
a substantially rigid matrix member disposed on said microprojection member top surface;
a compressible ring disposed on said microprojection member top surface and surrounding said rigid matrix member; and
a backing membrane disposed on said rigid matrix member and compressible ring.

28. The microprojection system of claim 27, wherein each of said plurality of stratum corneum-piercing microprojections has a length less than approximately 500 microns.

29. The microprojection system of claim 27, wherein each of said plurality of stratum corneum-piercing microprojections has a thickness in the range of approximately 5-50 microns.

30. The microprojection system of claim 27, wherein said rigid matrix and said compressible ring form a substantially planar disk.

31. The microprojection system of claim 27, wherein said compressible ring comprises a compressible foam.

32. The microprojection system of claim 31, wherein said foam comprises a material selected from the group consisting of polyethylene, polyurethane, neoprene, natural rubber, SPR, butyl, butadiene, nitrile, EPDM, ECH, polystyrene, polyester, polyether, polypropylene, EVE, EMA, metallocene resin, PVC, and blends thereof.

33. The microprojection system of claim 27, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

34. The microprojection system of claim 33, wherein each of said plurality of stratum corneum-piercing microprojections includes in the range of 1 microgram to 1 milligram of said biologically active agent.

35. A composite microprojection system, comprising:
a microprojection member having a top surface and a skin distal surface, said microprojection member including a plurality of stratum corneum-piercing microprojections that project from said skin distal surface, said microprojection member further including a reservoir containing at least one biologically active agent;
a substantially rigid matrix member disposed on said microprojection member top surface;
a compressible ring disposed on said microprojection member top surface and surrounding said rigid matrix member; and
a backing membrane disposed on said rigid matrix member and compressible ring.

36. The microprojection system of claim 35, wherein each of said plurality of stratum corneum-piercing microprojections has a length less than approximately 500 microns.

37. The microprojection system of claim 35, wherein each of said plurality of stratum corneum-piercing microprojections has a thickness in the range of approximately 5-50 microns.

38. The microprojection system of claim 35, wherein said rigid matrix and said compressible ring form a substantially planar disk.

39. The microprojection system of claim 35, wherein said compressible ring comprises a compressible foam.

40. The microprojection system of claim 39, wherein said foam comprises a material selected from the group consisting of polyethylene, polyurethane, neoprene, natural rubber, SPR, butyl, butadiene, nitrile, EPDM, ECH, polystyrene, polyester, polyether, polypropylene, EVE, EMA, metallocene resin, PVC, and blends thereof.

41. The microprojection system of claim 35, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

42. A composite microprojection system, comprising:
a microprojection member having a top surface and a skin distal surface, said microprojection member including a plurality of stratum corneum-piercing microprojections that project from said skin distal surface, said microprojection member further including an agent-containg matrix, said matrix including at least one biologically active agent;
a substantially rigid matrix member disposed on said microprojection member top surface;
a compressible ring disposed on said microprojection member top surface and surrounding said rigid matrix member; and
a backing membrane disposed on said rigid matrix member and compressible ring.

43. The microprojection system of claim 42, wherein said matrix is disposed proximate said top surface of said microprojection member.

44. The microprojection system of claim 42, wherein said matrix is disposed proximate said skin distal surface of said microprojection member.

45. The microprojection system of claim 42, wherein each of said plurality of stratum corneum-piercing microprojections has a length less than approximately 500 microns.

46. The microprojection system of claim 42, wherein each of said plurality of stratum corneum-piercing microprojections has a thickness in the range of approximately 5-50 microns.

47. The microprojection system of claim 42, wherein said rigid matrix and said compressible ring form a substantially planar disk.

48. The microprojection system of claim 42, wherein said compressible ring comprises a compressible foam.

49. The microprojection system of claim 48, wherein said foam comprises a material selected from the group consisting of polyethylene, polyurethane, neoprene, natural rubber, SPR, butyl, butadiene, nitrile, EPDM, ECH, polystyrene, polyester, polyether, polypropylene, EVE, EMA, metallocene resin, PVC, and blends thereof.

50. The microprojection system of claim 42, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

51. A composite applicator tip for an applicator, the applicator being adapted to apply a microprojection member, the applicator tip comprising:
a tip member adapted to cooperate with the applicator, said tip member having a skin distal surface; and
a compressible member disposed on said tip member skin distal surface.

52. The applicator tip of claim 51, wherein said tip member includes a substantially continuous recessed region on said tip member skin distal surface.

53. The applicator tip of claim, 52, wherein said compressible member is disposed in said recessed region.

54. The applicator tip of claim 51, wherein said compressible member comprises a compressible foam.

55. The applicator tip of claim 54, wherein said compressible foam comprises a substantially open-cell foam.

56. The applicator tip of claim 54, wherein said compressible foam comprises a substantially closed-cell foam.

57. The applicator tip of claim 54, wherein said foam comprises a material selected from the group consisting of polyethylene, polyurethane, neoprene, natural rubber, SPR, butyl, butadiene, nitrile, EPDM, ECH, polystyrene, polyester, polyether, polypropylene, EVE, EMA, metallocene resin, PVC, and blends thereof.

58. A composite applicator tip for an applicator, the applicator being adapted to apply a microprojection member, the applicator tip comprising:
a tip member adapted to cooperate with the applicator, said tip member having a skin distal surface, said tip member including a substantially continuous recessed region on said skin distal surface; and
a compressible member disposed in said recessed region.

59. The applicator tip of claim 58, wherein said recessed region is disposed proximate the outer periphery of said tip member skin distal surface.

60. The applicator tip of claim 58, wherein said compressible member comprises a compressible foam.

61. The applicator tip of claim 60, wherein said foam comprises a material selected from the group consisting of polyethylene, polyurethane, neoprene, natural rubber, SPR, butyl, butadiene, nitrile, EPDM, ECH, polystyrene, polyester, polyether, polypropylene, EVE, EMA, metallocene resin, PVC, and blends thereof.

62. A transdermal delivery system, comprising:
a microprojection member having a top surface and a skin distal surface, said microprojection member including a plurality of stratum corneum-piercing microprojections that project from said skin distal surface, a substantially rigid matrix member disposed on said microprojection member top surface, a compressible ring disposed on said microprojection member top surface and surrounding said rigid matrix member, and a backing membrane disposed on said rigid matrix member and compressible ring; and
an applicator adapted to apply said microprojection member, said applicator including an applicator tip that is adapted to contact said microprojection member when said applicator is employed to apply said microprojection member.

63. The delivery system of claim 62, wherein each of said plurality of stratum corneum-piercing microprojections has a length less than approximately 500 microns.

64. The delivery system of claim 62, wherein each of said plurality of stratum corneum-piercing microprojections has a thickness in the range of approximately 5-50 microns.

65. The delivery system of claim 62, wherein said compressible ring comprises a compressible foam.

66. The delivery system of claim 65, wherein said foam comprises a material selected from the group consisting of polyethylene, polyurethane, neoprene, natural rubber, SPR, butyl, butadiene, nitrile, EPDM, ECH, polystyrene, polyester, polyether, polypropylene, EVE, EMA, metallocene resin, PVC, and blends thereof.

67. The delivery system of claim 62, wherein said microprojection member is coated with a biocompatible coating, said biocompatible coating including at least one biologically active agent.

68. The delivery system of claim 67, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

69. The delivery system of claim 67, wherein each of said plurality of stratum corneum-piercing microprojections includes in the range of 1 microgram to 1 milligram of said biologically active agent.

70. The delivery system of claim 62, wherein said microprojection member includes a reservoir.

71. The delivery system of claim 70, wherein said reservoir includes at least one biologically active agent.

72. The delivery system of claim 71, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

73. The delivery system of claim 62, wherein said microprojection member includes an agent-containing matrix.

74. The delivery system of claim 73, wherein said matrix is disposed proximate said top surface of said microprojection member.

75. The delivery system of claim 73, wherein said matrix is disposed proximate said skin distal surface of said microprojection member.

76. The delivery system of claim 73, wherein said matrix includes at least one biologically active agent.

77. The delivery system of claim 76, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

78. A transdermal delivery system, comprising:
a microprojection member having a top surface and a skin distal surface, said microprojection member including a plurality of stratum corneum-piercing microprojections that project from said skin distal surface of said microprojection member; and
an applicator adapted to apply said microprojection member, said applicator including an applicator tip having a skin distal surface that is adapted to contact said microprojection member when said applicator is employed to apply said microprojection member, said applicator tip including a compressible member disposed on said skin distal surface of said applicator tip.

79. The delivery system of claim 78, wherein said applicator tip includes a substantially continuous recessed region on said skin distal surface of said applicator tip.

80. The delivery system of claim 79, wherein said compressible member is disposed in said recessed region.

81. The delivery system of claim 78, wherein said compressible member comprises a compressible foam.

82. The delivery system of claim 81, wherein said foam comprises a material selected from the group consisting of polyethylene, polyurethane, neoprene, natural rubber, SPR, butyl, butadiene, nitrile, EPDM, ECH, polystyrene, polyester, polyether, polypropylene, EVE, EMA, metallocene resin, PVC, and blends thereof.

83. The delivery system of claim 78, wherein said microprojection member is coated with a biocompatible coating, said biocompatible coating including at least one biologically active agent.

84. The delivery system of claim 83, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

85. The delivery system of claim 83, wherein each of said plurality of stratum corneum-piercing microprojections includes in the range of 1 microgram to 1 milligram of said biologically active agent.

86. The delivery system of claim 78, wherein said microprojection member includes a reservoir.

87. The delivery system of claim 86, wherein said reservoir includes at least one biologically active agent.

88. The delivery system of claim 87, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

89. The delivery system of claim 78, wherein said microprojection member includes an agent-containing matrix.

90. The delivery system of claim 89, wherein said matrix is disposed proximate said top surface of said microprojection member.

91. The delivery system of claim 89, wherein said matrix is disposed proximate said skin distal surface of said microprojection member.

92. The delivery system of claim 89, wherein said matrix includes at least one biologically active agent.

93. The delivery system of claim 92, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

94. A transdermal delivery system, comprising:
a microprojection member having a top surface and a skin distal surface, said microprojection member including a plurality of stratum corneum-piercing microprojections that project from said skin distal surface of said microprojection member, a substantially rigid matrix member disposed on said microprojection member top surface, a first compressible member disposed on said microprojection member top surface and surrounding said rigid matrix member, and a backing membrane disposed on said rigid matrix member and first compressible member; and
an applicator adapted to apply said microprojection member, said applicator including an applicator tip having a skin distal surface that is adapted to contact said microprojection member when said applicator is employed to apply said microprojection member, said applicator tip including a second compressible member disposed on said skin distal surface of said applicator tip.

95. The delivery system of claim 94, wherein each of said plurality of stratum corneum-piercing microprojections has a length less than approximately 500 microns.

96. The delivery system of claim 94, wherein each of said plurality of stratum corneum-piercing microprojections has a thickness in the range of approximately 5-50 microns.

97. The delivery system of claim 94, wherein said applicator tip includes a substantially continuous recessed region on said skin distal surface of said applicator tip.

98. The delivery system of claim 97, wherein said second compressible member is disposed in said recessed region.

99. The delivery system of claim 94, wherein said first and second compressible members comprise a compressible foam.

100. The delivery system of claim 99, wherein said foam comprises a material selected from the group consisting of polyethylene, polyurethane, neoprene, natural rubber, SPR, butyl, butadiene, nitrile, EPDM, ECH, polystyrene, polyester, polyether, polypropylene, EVE, EMA, metallocene resin, PVC, and blends thereof.

101. The delivery system of claim 94, wherein said microprojection member is coated with a biocompatible coating, said biocompatible coating including at least one biologically active agent.

102. The delivery system of claim 101, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

103. The delivery system of claim 101, wherein each of said plurality of stratum corneum-piercing microprojections includes in the range of 1 microgram to 1 milligram of said biologically active agent.

104. The delivery system of claim 94, wherein said microprojection member includes a reservoir.

105. The delivery system of claim 104, wherein said reservoir includes at least one biologically active agent.

106. The delivery system of claim 105, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.

107. The delivery system of claim 94, wherein said microprojection member includes an agent-containing matrix.

108. The delivery system of claim 107, wherein said matrix is disposed proximate said top surface of said microprojection member.

109. The delivery system of claim 107, wherein said matrix is disposed proximate said skin distal surface of said microprojection member.

110. The delivery system of claim 107, wherein said matrix includes at least one biologically active agent.

111. The delivery system of claim 110, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM,-CSF, G-CSF, IL-10, glucagon, growth hormone releasing factor (GRF) and analogs thereof, including pharmaceutically acceptable salts, conventional vaccines, recombinant protein vaccines, DNA vaccines and therapeutic cancer vaccines.
